(19) United States
(12) Patent Application Publication
(10) Pub. No.: US 2025/0144406 A1
(43) Pub. Date: May 8, 2025

(54) ELECTRICAL MUSCLE STIMULATION APPARATUS AND METHODS

(71) Applicant: LF Bolt Corp., Costa Mesa, CA (US)

(72) Inventor: Colleen McNUTT, Costa Mesa, CA (US)

(21) Appl. No.: 18/730,299

(22) PCT Filed: Jan. 18, 2023
(86) PCT No.: PCT/US2023/060860

§ 371 Date: Jul. 18, 2024

Related U.S. Application Data
(60) Provisional application No. 63/300,609, filed on Jan. 18, 2022.

(51) Int. Cl. A61N 1/04; A61N 1/36

(52) U.S. Cl. CPC A61N 1/0484; A61N 1/36003; A61N 1/36031; A61N 1/36034

(57) ABSTRACT

Described herein are electrical muscle stimulation (EMS) apparatuses (e.g., devices and systems, including suits, controls, etc.). These EMS apparatuses may include an integrated controller and power supply, including an integrated controller and power supply with a user input/output (e.g., touch-screen) that is compact and may be coupled to an EMS suit for controlling the suit and/or for communicating with one or more remote servers. Also described herein are EMS apparatuses having wettable electrical contacts that are adapted for reliable and easy use by the wearer of the suit (e.g., in an at-home or studio setting). Also described herein are EMS configured to enhance the safety of operation and effectiveness of EMS.

### CLAIM OF PRIORITY

This patent application claims priority to U.S. provisional patent application No. 63/300,609, filed Jan. 18, 2022, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Electrical Muscle Stimulation (EMS) elicits muscle contraction using electrical impulses. The impulses are delivered via electrodes placed on the body near the muscles that are to be stimulated. EMS technology has been incorporated into fitness products, such as EMS suits, which may help users achieve their health and fitness goals, such as burning calories, improving muscle tone, increasing strength, and/or recovering from an injury. Electrodes in the EMS suit may be situated near particular muscles groups (e.g., arms, legs, chest, abdominals, back, etc.) in order to deliver electric impulses targeted to those muscle groups while the user performs various exercise movements.

Existing EMS technology may be coordinated with a defined workout plan or instructor and a user of an EMS suit may coordinate training with an instructor or with a prerecorded class. However currently available EMS systems may be difficult to operate, particularly when operated in an at-home or studio environment. For example, a user may have difficulty making reliable connection between the electrodes and the EMS suit.

In addition, EMS suits may be difficult for the user to apply and control. There are a number of complex parameters possible when controlling an EMS apparatus, including an EMS suit, related to the intensity (e.g., peak current amplitude), frequency, pulse width and the like, as well as the physical characteristics of the electrodes and the user's skin (e.g., impedance, skin conductance, etc.). Users may not be able to synthesize these complex parameters, and may benefit from a simple, easy-to-use and reliable user interface with the EMS apparatus and/or the workouts to be used.

In addition, there are a number of safety concerns when using EMS suits and methods. For example, overuse of EMS, or use of EMS at higher intensities, or at intensities that are ill-matched to the condition of the user may be uncomfortable or even painful and may prevent treatment gains.

The methods and apparatuses described herein may address these issues.

### SUMMARY OF THE DISCLOSURE

Described herein are methods and apparatuses (including EMS suits, user interfaces, and control systems, etc.), which may include hardware, software and/or firmware, for electrical muscle stimulation (EMS) systems that may provide safer, easier to use, and more reliable EMS than previously available. In general the apparatuses and methods described herein may be used as part of a therapeutic or non-therapeutic procedure. For example, these methods and apparatuses may be part of an exercise or fitness (including weight loss) regime. However, the methods and apparatuses described herein may also or alternatively be used as part of a therapy, such as in particular for physiotherapy and/or for treatment of a condition such as restless leg syndrome (RLS), neuromuscular disorders (including, but not limited to Parkinson's, Amyotrophic lateral sclerosis (ALS), etc.).

For example, described herein are EMS suit apparatuses and method of using them. For example, an EMS suit apparatus may include a plurality of electrode assemblies that are configured to hold a conductive fluid so that the electrode assemblies may make consistent and reliable electrical contact with the user's skin. These electrode assemblies may include a port for allowing wetting of the electrode assemblies, such as a fluid reservoir of the electrode assembly the fluid reservoir may include a porous region.

For example, described herein are electrical muscle stimulation (EMS) apparatuses (e.g., suits) comprising: a garment comprising an elastic material that is configured to conform to a user's body; a plurality of electrode assemblies on an inner face of the garment, the electrode assemblies each configured to sit flush against the user's skin; a plurality of sensors distributed on, in, or over the garment; a plurality of electrical connectors electrically coupling the electrode assemblies to controller for applying EMS; and a controller configured to control the application of energy to individual electrodes assemblies of the plurality of electrode assemblies, wherein the controller is configured to receive sensor data from the plurality of sensors and to identify a type of exercise being performed by the user, and to set a subset of the plurality of electrode assemblies to activate based on the identified type of exercise.

The controller may comprise a trained machine learning agent configured to identify the type of exercise being performed by the user. The controller may be further configured to recommend or adjust the energy applied by the subset of the plurality of electrode assemblies based on the type of exercise identified, and/or the sensor input and/or based on user-specific data. In some examples, the controller includes a trained machine learning agent configured to recommend or adjust the energy applied by the subset of the plurality of electrode assemblies and based on sensor data from the plurality of sensors. In some examples the controller includes a trained machine learning agent configured to recommend or adjust the energy applied by the subset of the plurality of electrode assemblies and based on user-specific data. The user-specific data may comprise one or more of: user past performance, user height, user weight, user BMI, user physical condition, user age, user biometric data from the plurality of sensors.

Any of these garments may include one or more of: a shirt, vest, pants, or shorts. In any of these apparatuses and methods the plurality of sensors may comprise one or more of: motion sensors, bioimpedance sensors, and position sensors.

Also described herein are electrical muscle stimulation (EMS) apparatuses including: a garment comprising an elastic material that is configured to conform to a user's body; a plurality of electrode assemblies on an inner face of the garment, the electrode assemblies each configured to sit flush against the user's skin; a plurality of sensors distributed on, in, or over the garment; a plurality of electrical connectors electrically coupling the electrode assemblies to controller for applying EMS; and a controller configured to control the application of energy to individual electrodes assemblies of the plurality of electrode assemblies, wherein the controller comprises a trained machine learning agent configured to recommend and/or adjust the energy applied by the plurality of electrode assemblies based on data from the plurality of sensors. The controller may be configured to identify the type of exercise being performed by the user based on the plurality of sensors. The trained machine learning agent may be configured to recommend or adjust the energy applied by the subset of the plurality of electrode assemblies based on user-specific data and exercise type. For example, the controller may be configured to automatically or semi-automatically adjust the energy applied by the plurality of electrode assemblies based on the recommendation of the trained machine learning agent.

The trained machine learning agent may be configured to recommend or adjust the energy applied by the subset of the plurality of electrode assemblies based on data from the plurality of sensors and based on user-specific data. The user-specific data may be one or more of: user past performance, user height, user weight, user BMI, user physical condition, user age, user biometric data from the plurality of sensors.

Also described herein are methods and apparatuses for determining compliance of EMS apparatuses. For example, an electrical muscle stimulation (EMS) suit apparatus may include: a garment comprising an elastic material that is configured to conform to a user's body; a plurality of electrode assemblies on an inner face of the garment, the electrode assemblies each configured to sit flush against the user's skin; a plurality of electrical connectors electrically coupling the electrode assemblies to controller for applying EMS; and a controller comprising a compliance monitoring module, where the compliance monitoring module is configured to track the application of energy to the user's skin over a compliance period and to output compliance data based.

An electrical muscle stimulation (EMS) suit apparatus may include: a garment comprising an elastic material that is configured to conform to a user's body; a plurality of electrode assemblies on an inner face of the garment, the electrode assemblies each configured to sit flush against the user's skin; a plurality of electrical connectors electrically coupling the electrode assemblies to controller for applying EMS; and a controller comprising a compliance monitoring module, where the compliance monitoring module is configured to track the verified application of energy to the user's skin over a compliance period and to output compliance data based, wherein the verified application of energy to the user's skin is based on bioimpedance determined from one or more of the electrode assemblies.

Described herein are electrical muscle stimulation (EMS) suit apparatuses that include: an upper torso region comprising an elastic material that is configured to conform to a user's torso; a plurality of electrode assemblies on an inner face of the upper torso region, the electrode assemblies each comprising an electrically conductive base that is adjacent to a porous skin-contacting region, wherein the porous skin-contacting region is configured to hold a conductive fluid within the pores and an inlet port in fluid communication with the porous skin-contacting region; and a plurality of electrical connectors electrically coupling the electrically conductive base of each of the electrode assemblies to a coupler region configured to be electrically coupled to a controller for applying EMS.

The inlet ports of the plurality of electrode assemblies may extend from an outer face of the upper torso region. Thus, in some examples the electrode assemblies may be wetted by the user or for the user without removing the apparatus. In some examples the apparatus does not include a port, but the skin-contacting surface is wetted directly.

The porous skin-contacting region may comprise a sponge material (e.g., a compliant porous material), a hydrogel, etc. In some examples, the assembly includes a separate reservoir. Each of the plurality of electrode assemblies may comprise a sensor configured to detect fluid within the porous skin-contacting region.

The inlet port may be configured to receive a spray nozzle.

Any of these apparatuses may also include a lower region that is configured to confirm the user's legs and buttocks.

In any of these apparatuses, the apparatus may include a controller, wherein the controller is configured to be secured to the EMS suit apparatus. The controller may be a hybrid power source/controller. The controller may be configured to dynamically adjust a user-specific maximum EMS power applied during a treatment session. For example, the controller may be configured to wirelessly communicate with one or more remote processors to receive EMS treatment parameters.

Any of these apparatuses may include a pocket that is configured to receive the controller. The pocket may be on the upper or lower regions.

For example, an electrical muscle stimulation (EMS) suit apparatus may include: an upper torso region comprising an elastic material that is configured to conform to a user's torso; a plurality of electrode assemblies on an inner face of the upper torso region, the electrode assemblies each comprising an electrically conductive base that is adjacent to a porous skin-contacting region, wherein the porous skin-contacting region is configured to hold a conductive fluid within the pores and an inlet port extending to an outer face of the upper torso region that is in fluid communication with the porous skin-contacting region configured to receive the conductive fluid for delivery to the porous skin-contacting region; a plurality of electrical connectors electrically coupling the electrically conductive base of each of the electrode assemblies to a coupler region configured to be electrically coupled to a controller; and a controller configured to couple with couple region, wherein the controller is configured to apply EMS between pairs of electrode assemblies.

Also described herein are electrical muscle stimulation (EMS) suit apparatuses comprising: an upper torso region that is configured to conform to a user's torso; a plurality of electrode assemblies on an inner face of the upper torso region; and a plurality of electrical connectors electrically coupling each of the electrode assemblies to a coupler region configured to be electrically coupled to a controller for applying EMS; and a combined power source and controller configured to couple with couple region, wherein the controller is configured to apply EMS between pairs of electrode assemblies, wherein the combined power source and controller comprises a display screen and one or more inputs for selecting an EMS applied power level.

The display screen may comprise a touchscreen. In any of these apparatuses the apparatus may include an override shutoff control (e.g., on the combined power source and controller) that is configured to shut down power to the plurality of electrode assemblies.

The upper torso region may include an elastic or stretch material configured to confirm to the user's torso. In some examples the elastic material is a polymeric material, such a wetsuit material (e.g., a neoprene material).

Any of these apparatuses may include a lower region that is configured to confirm the user's legs and buttocks.

In general, the combined power source and controller may be configured to be secured to the EMS suit apparatus so that the display screen may be viewed by the user. The combined power source and controller may be configured to dynamically adjust a user-specific maximum EMS power applied during a treatment session. The combined power source and controller may be configured to wirelessly communicate with one or more remote processors to receive EMS treatment parameters.

Any of these apparatuses may include a pocket on the upper torso region that is configured to receive the combined power source and controller.

For example, described herein are methods of electrical muscle stimulation (EMS) including: determining an initial baseline EMS power for a user; determining recent EMS applied to the user within a predetermined time period; estimating a user-specific maximum EMS power based on the initial baseline EMS power and on the recent EMS applied to the user within the predetermined time period; applying EMS to the user during a treatment session, wherein the applied EMS is a percentage of the user-specific maximum EMS power that increases over a duration of the treatment session from an initial minimum percentage to a maximum of 100% of the user-specific maximum EMS power.

The initial baseline EMS power may be a function of one or more of: current amplitude, frequency, and pulse width. Determining the initial baseline EMS power for the user may comprise receiving the initial baseline EMS power from a data storage holding the initial baseline EMS power. In some examples determining the initial baseline EMS power for the user comprises calculating the initial baseline EMS power for the user from user-provided self-reporting data. For example, determining recent EMS applied to the user within the predetermine time period may comprise determining recent EMS applied to the user within the last 10 days or less.

Estimating the user-specific maximum EMS power may include estimating user-specific maximum EMS powers for each of a plurality of pairs of electrode of an EMS apparatus corresponding to the user. For example, estimating the user-specific maximum EMS power may comprise increasing the user-specific maximum EMS power based on a number of treatment sessions within the predetermined time period. Estimating the user-specific maximum EMS power may comprise determining immediately before a new treatment session is begun.

Applying EMS to the user during the treatment session may comprise increasing one or more of: current amplitude, frequency, and pulse width of the applied EMS. For example, the user-specific maximum EMS power may decrease as the time from the most recent EMS applied to the user increases.

For example, a method of electrical muscle stimulation (EMS) may include: determining an initial baseline EMS power for a user, wherein the initial baseline EMS power is a function of one or more of: current amplitude, frequency, and pulse width; determining recent EMS applied to the user within a predetermined time period of 10 days or less; estimating a user-specific maximum EMS power based on the initial baseline EMS power and on the recent EMS applied to the user within the predetermined time period, wherein the user-specific maximum EMS power decreases as the time from the most recent EMS applied to the user increases; applying EMS to the user during a treatment session, wherein the applied EMS is limited to a percentage of the user-specific maximum EMS power that increases over a duration of the treatment session from an initial minimum percentage to a maximum of 100% of the user-specific maximum EMS power.

Also described herein are methods of electrical muscle stimulation (EMS) that include: determining an initial baseline EMS power for a user; determining recent EMS applied to the user within a predetermined time period of more than 2 days; estimating, before initiating a treatment session, a user-specific maximum EMS power based on the initial baseline EMS power and on the recent EMS applied to the user within the predetermined time period, wherein the user-specific maximum EMS power is set to zero if the user has received a minimum EMS treatment within 40 hours or less, otherwise estimating the user-specific maximum EMS power from the initial baseline EMS power and the recent EMS applied to the user within the predetermined time period; and applying EMS to the user during the treatment session if the user-specific maximum EMS power is greater than zero.

The initial baseline EMS power may be a function of one or more of: current amplitude, frequency, and pulse width. Determining the initial baseline EMS power for the user may comprise receiving the initial baseline EMS power from a data storage holding the initial baseline EMS power. Determining the initial baseline EMS power for the user may comprise calculating the initial baseline EMS power for the user from user-provided self-reporting data. In some examples determining recent EMS applied to the user within the predetermine time period comprises determining recent EMS applied to the user within between the last 2-10 days.

Estimating the user-specific maximum EMS power may include estimating user-specific maximum EMS powers for each of a plurality of pairs of electrode of an EMS apparatus corresponding to the user. If the user-specific maximum EMS power is set to zero, any of these methods may include alerting the user that it has been too recent to allow EMS. In some examples, applying EMS to the user during the treatment session comprises increasing one or more of: current amplitude, frequency, and pulse width of the applied EMS.

The user-specific maximum EMS power may decrease as the time from the most recent EMS applied to the user increases.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIGS. 1A-1F show one example of an EMS apparatus as described herein.

FIGS. 2A-2C show an example of parts of an EMS apparatus.

FIGS. 3A-3B illustrate examples of EMS apparatuses (e.g., EMS suits) worn on a user.

FIGS. 4A-4B show another example of an EMS apparatus as described herein.

FIG. 5 illustrates hydrating an example of an electrode for an EMS apparatus.

FIG. 6 shows one example of a power supply for an EMS apparatus.

FIGS. 7A-7D illustrate an example of a combined power supply and controller for an EMS apparatus as described herein.

FIGS. 8A-8E illustrate an example of a combined power supply and controller for an EMS apparatus.

FIGS. 9A-9F illustrate an example of a combined power supply and controller for an EMS apparatus.

FIG. 10 illustrates one example of a portion of a combined power supply/controller for an EMS apparatus.

FIG. 11 schematically illustrates one example of an EMS apparatus as described herein.

FIG. 12 schematically illustrates an example of a system (e.g., including application software) for an EMS apparatus.

FIG. 13 schematically illustrates one example of a control subsystem as described herein.

FIG. 14 is a schematic of one example of a method for automatically or semi-automatically adjusting electrode parameters using a trained machine learning agent as described herein.

FIG. 15 schematically illustrates an example of a method of determining compliance when operating an EMS apparatus.

### DETAILED DESCRIPTION

Described herein are electrical muscle stimulation (EMS) apparatuses (e.g., devices and systems, including suits, controls, etc.). These EMS apparatuses may include an integrated controller and power supply, including an integrated controller and power supply with a user input/output (e.g., touchscreen) that is compact and may be coupled to an EMS suit for controlling the suit and/or for communicating with one or more remote servers. Also described herein are EMS apparatuses having wettable electrical contacts that are adapted for reliable and easy use by the wearer of the suit (e.g., in an at-home or studio setting). Also described herein are EMS suits that are more comfortable and easier to put on and take off (particularly for user's that may be impaired), including EMS suits that include electrodes that are flush with inside of the suit and that do not include external cabling. These EMS suit apparatuses may be easier to put on, adjust and maneuver in than traditional EMS suits, and may allow movement and flexibility while maintaining reliable and sufficient contact between the user and the multiple EMS electrodes. Any of the apparatuses described herein may include a user interface configured to enhance the ease of operation and effectiveness of an EMS suit. For example, described herein are apparatuses (e.g., systems) that may be used to regulate the safe and effective operation of the EMS suit, including limiting or preventing operation in ways that may be less effective and/or dangerous to the user. Any of the apparatuses (e.g., EMS suits, systems including them, etc.) and methods described herein may include control logic that includes a trained neural network for determining one or more of: a condition of the user (e.g., a fitness level, an impairment, etc.), a movement (e.g., exercise, routine, sequence of positions, etc.) of the user, and/or fitness or therapeutic goals for the user, and adjusting or controlling the EMS applied by the suit.

Any of the apparatuses and methods described herein may also or alternatively be configured to determine and report compliance of a user.

In general, an EMS apparatus may include an EMS suit having a plurality of electrodes coupled or couplable thereto, wherein the electrodes are positioned/positionable on the EMS suit in an arrangement that provide muscle stimulation while preventing dangerous (e.g., transthoracic flow) of electrical current through a body of a user wearing the EMS suit during operation of the EMS suit. Individual electrodes of the EMS suit may be controllable by a processor(s) to deliver electrical impulses to muscles of a user who is wearing the EMS suit. When an electrical impulse is delivered via a pair of electrodes, electrical current (i.e., the flow of charged particles) flows from one electrode (of the pair), through a portion of the user's body (e.g., through muscle tissue underlying the pair of electrodes), and to the other electrode (of the pair). The user's body completes an electrical circuit that includes the pair of electrodes, thereby allowing electrical current to flow between the pair of electrodes during operation of the EMS suit, as electrical impulses are delivered via the electrodes. A pair of electrodes may include two electrodes that correspond to a common channel of multiple channels that are used to deliver electrical impulses, channel-by-channel, during operation of the EMS suit. A pair of electrodes may also include two electrodes that allow for electrical current to flow therebetween during operation of the EMS suit, one electrode of the pair operating as a positive electrode (anode) and the other electrode of the pair operating as a negative electrode (cathode). With alternating current (AC), each electrode of a given pair may reverse current with each cycle (or frame). That is, each electrode may change from a positive electrode (anode) to a negative electrode (cathode) with each cycle (or frame).

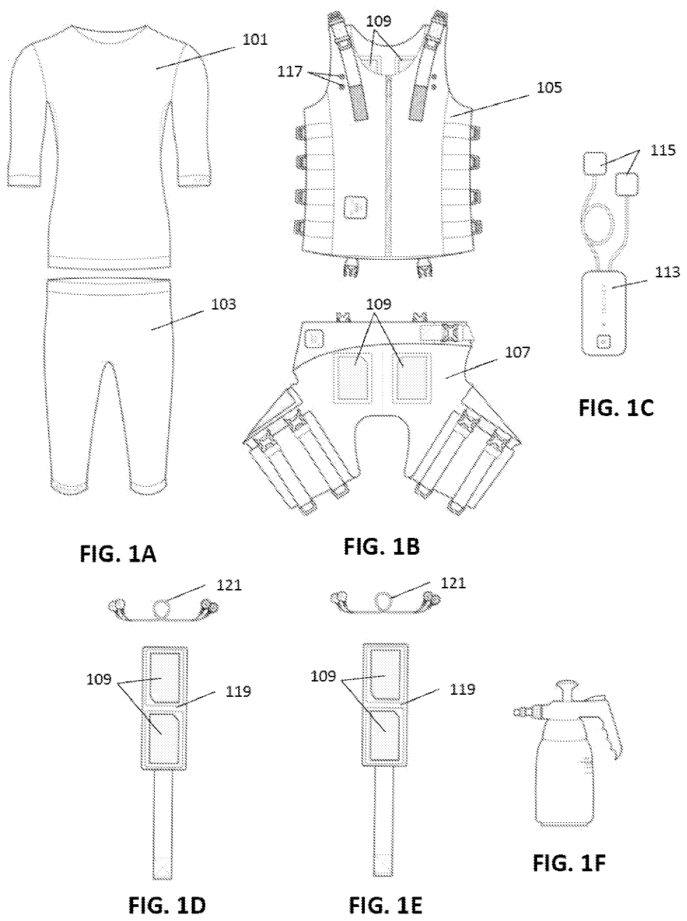

For example, FIGS. 1A-1F illustrate an example of an EMS system as described herein. This example shows a wireless, whole-body electrical muscle stimulation (EMS) system that includes a suit/vest, a lower body (pants/shorts) portion, a combined power supply/controller/user interface, and an electrode wetting source (e.g., spray bottle). FIG. 1A shows an example of an upper 101 and lower 103 under suit. The under suit may be configured to allow electrical connection between electrodes and the underlying skin in the appropriate region of the body (e.g., over the target muscle groups). For example, the under suit may include openings and/or electrically conductive regions (or may be wholly conductive). The under suit may be configured to conform to the patient's body, e.g., as a stretch and/or compression garment. The under suit may be washable.

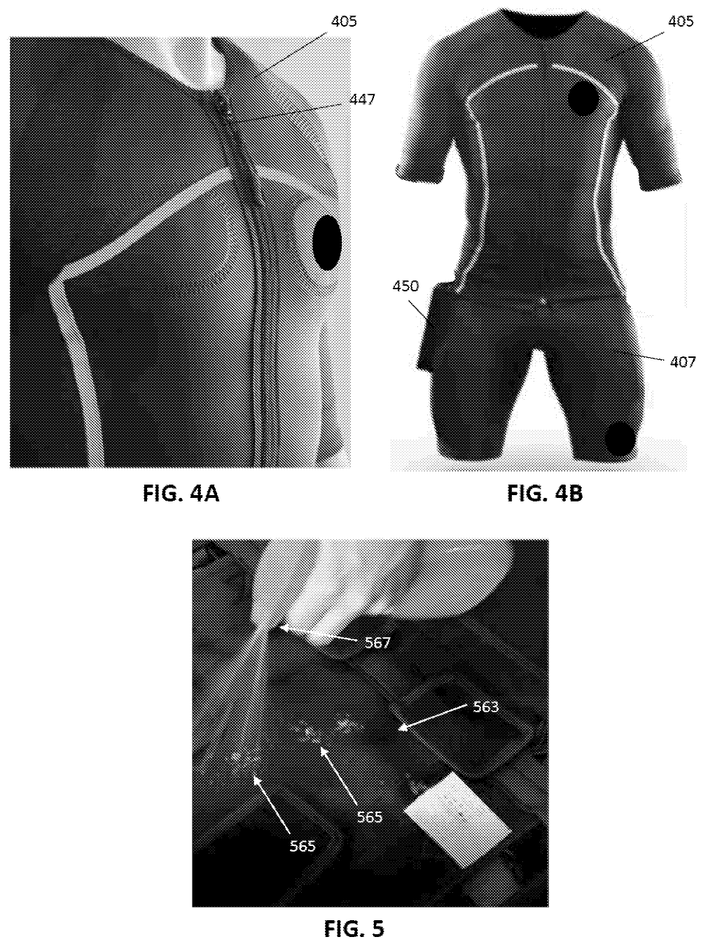

FIG. 1B shows an example of an upper torso (e.g., vest) portion 105 of the EMS suit and a lower body 107 portion of the EMS suit. The upper torso portion and the lower body portion may support the plurality of electrodes 109, which may be integrated into the apparatus. These electrodes, as described in greater detail below, may be wettable electrodes adapted to be easily wetted by the user. The upper torso and lower body portions may include one more adjustable straps allowing the user to attach and adjust the fit. The upper torso 105 portion shown in FIG. 1B is configured as a vest, and the lower body region is configured as a chaps-like configuration to be worn over the under suit. In some examples the under suit and the upper torso and lower body regions may be integrated together into a single garment, as shown in FIGS. 4A-4B, below.

In any of these examples the EMS suit may have electrodes strategically positioned so as to apply EMS to the target muscle groups, such as the quadriceps (quads), hamstrings, glutes, abs, chest, lower back, mid back, upper back (trapezius), biceps and triceps, and/or calves.

In any of these examples, the electrical connectors (e.g., “cables”) may be integrated into the suit. For example, coupling the power supply/controller into the suit may automatically couple the electrodes to the power source/controller via a single (e.g., multiplexed) connection, dramatically simplifying the contact. For example, the integrated electrical connectors may be coupled via internal cabling that is arranged so as not to limit freedom of movement.

In some examples the cables are covered by a vinyl material that may be sealed (e.g., thermally sealed) to the body of the garment, which may be spandex/elastane, e.g., Lycra or other highly conforming material. The cables may be sealed to the garment with regions that are less stretchable, in which the cabling runs in a straight line, with little or no slack, between regions (e.g., near joints or body regions that vary greatly by user body type, such as the chest/bust region and the pannus) as that are more stretchable, in which the cabling may be permitted to slide or move relative to the material surrounding the cabling (e.g., vinyl and/or spandex).

The electrodes of the EMS suit may be arranged in the EMS suit to cover parts of the user's body in order to excite particular muscle groups (e.g., arms, legs, chest, abdominals, back, etc.) through the delivery of electrical impulses that stimulate the muscle tissue beneath the user's skin. In particular, as will be described in greater detail below, the electrodes described herein may be arranged in a manner that increases the ability of the electrodes to remain in reliable communication with the patient's skin and therefore provide energy to the underlying muscle during a treatment.

For example, the upper body portion (e.g., torso, including chest, vest, etc.) may be worn on an upper trunk of the user's body. The upper body portion may be coupled to the lower body portion, e.g., via one or more mechanical and/or electrical connectors. Thus any of the buckles/straps shown may include both mechanical and electrical connectors.

In some of these apparatuses the connectors 111 (e.g., buckles) may be configured to make and/or confirm electrical connection. For example, the power source/controller may sense and/or confirm that each connector is coupled and/or secured. The controller may, for example, provide a test current/pulse to confirm the electrical connection (via. the electrical properties of the connection, showing an open circuit if not properly attached). The electrical contact with the skin of the user may similarly or additionally be confirmed by the system and may be used as part of a safter interlock and/or power-saving protocol.

In some examples the upper body portion may comprise a left front portion, a right front portion, and a back portion. In some embodiments, the left front portion has a first pair of electrodes positioned on an inner surface of the left front portion and within a top half of the left front portion, while the right front portion has a second pair of electrodes positioned on an inner surface of the right front portion and within a top half of the right front portion. In this manner, when the body of a user is wearing the EMS suit, the first pair of electrodes may be disposed on (or atop) one or more left pectoral muscles of the body, and the first pair of electrodes may, therefore, be positioned on a first (e.g., left) side of a midsagittal plane of the body, as well as on a first (e.g., front) side of the frontal plane of the body. A second pair of electrodes may be disposed on (or atop) one or more right pectoral muscles of the body, and the second pair of electrodes may be positioned on a second (e.g., right) side of the midsagittal plane of the body, as well as on the first (e.g., front) side of the frontal plane of the body.

Electrical impulses may be delivered by the respective pairs of electrodes so that the flow of electrical current is primarily passed between each pair of electrodes, though the target muscle region, so that electrical current does not flow between an electrode of the first pair of electrodes and an electrode of the second pair of electrodes, or any other pair, in order to avoid applying electrical energy to regions that are not intended to be stimulated. For example, the apparatuses described herein may prevent electrical current from flow across the midsagittal plane of the user's body in a region of the thoracic cavity during operation of the EMS suit.

FIG. 1C also illustrates an example of an integrated controller/power supply 113. In this example, the integrated power supply/controller includes a separate mechanical and electrical connector; however in some examples the same connector may make both mechanical and electrical connection with the EMS suit. For example, in FIG. 1C the apparatus includes a pair of electrical connectors 115 that may attach to electrical coupling contacts on the suit. In FIGS. 1A-IF only a single pair (e.g., anode/cathode) of connectors are shown. In some examples, multiple connectors/contacts may be used. In this example the suit may include an integrated multiplexing electrical manifold that may direct and/or switch the applied energy to the one or more pairs of electrodes to which power is to be applied to apply EMS. In general, power may be applied to individual pairs of electrodes at a time (sequentially) or in a manner so that nearby electrodes in communication with the body are not concurrently activated by the application of electrical energy. The power supply/controller 113 may be held to the EMS suit by a pocket and/or a mechanical connector such as Velcro, straps, etc. Multiple mechanical connectors may be included.

FIGS. 1D and 1E illustrate examples of arm electrode supports 119 including electrodes 109 that may be used to apply electrical energy to the biceps and/or triceps. In this example, in which the arm electrode supports are not integral with the upper/torso portion 105, addition external cables 121 may be used to connect the arm electrodes to contact 117 on the upper/torso portion (vest) 105. Alternatively in some examples the electrode and/or electrode supports may be integrated into an upper/torso EMS garment.

In any of these apparatuses, the back of the upper body portion of the EMS suit may include multiple electrodes 109, including, e.g., a third pair of electrodes positioned on an inner surface of the back portion and within a left half of the back portion, and a fourth pair of electrodes positioned on the inner surface of the back portion and within a right half of the back portion. When the body of a user is wearing the EMS suit, the third pair of electrodes may be disposed on (or atop) one or more left back muscles of the body, and the third pair of electrodes may, therefore, be positioned on the first (e.g., left) side of a midsagittal plane of the body, as well as on a second (e.g., back) side of the frontal plane of the body. Meanwhile, the fourth pair of electrodes may be disposed on (or atop) one or more right back muscles of the body, and the fourth pair of electrodes may, therefore, be positioned on the second (e.g., right) side of the midsagittal plane of the body, as well as on the second (e.g., back) side of the frontal plane of the body. Other configurations and arrangements may be used.

Any of the electrodes described herein may be wettable electrodes that include an absorbent substrate (forming a fluid/wetting reservoir) in electrical communication with the electrode and configured to contact the user's skin (either directly or through the under suit). These wettable electrodes are configured to hold a conductive fluid (e.g., water, including saline) that may help make a reliable electrical contact with the user's skin, and maintain the electrical properties, even as the user sweats during physical exercise wearing the EMS suit. This may be particularly helpful, as these wettable electrodes may be configured (by size and position) to allow continuous electrical contact with the skin without having the electrical properties significantly change doe to sweating. The electrode assembly may also include one or more ports or openings configured to mate with the fluid source 125 (e.g., such as the spray bottle shown as an example in FIG. 1F). to allow delivery of the conductive fluid (e.g., saline) to the fluid reservoir. For example, each electrode assembly may include a port on an outward-facing side to which the fluid source may be engaged to apply (e.g., by spraying) fluid. The fluid port may also be configured to prevent leakage of fluid (e.g., saline) out of the port and/or out of the electrode assembly. The electrode assembly may include the fluid reservoir, which may be a porous material (e.g., sponge and/or wettable hydrogel, etc.).

The apparatus shown in FIGS. 1A-IF does not show the associated application or other components of the apparatus that may be used to control the applied power to drive EMS of target muscle(s). The controller 113 may include a user interface (e.g., touchscreen) for direct communication with the user and/or it may be configured to wirelessly communicate with one or more external processors, such as a smartphone, tablet, computer, etc. In some examples the user may communicate via a smartphone or tablet (not shown). In some examples the user may communicate with a remote processor. Any of all of the controller/power supply, smartphone, and/or remote processor may include software, firmware and/or hardware for engaging with the user and/or for controlling operation of the apparatus, including for engaging in one or more safety protocols to prevent a user from exceeding a predetermined or calculated amount of EMS to individual body regions (muscled) and/or the entire body based on the user's condition and prior operation of one or more EMS apparatuses.

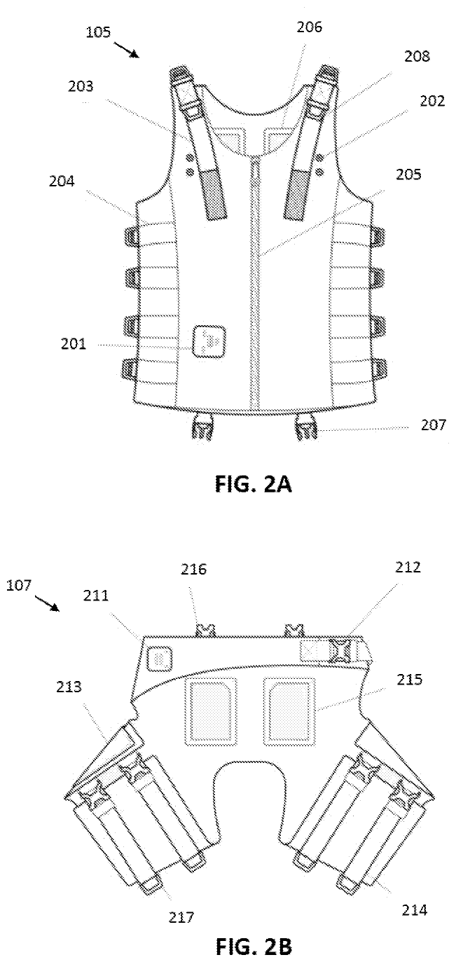

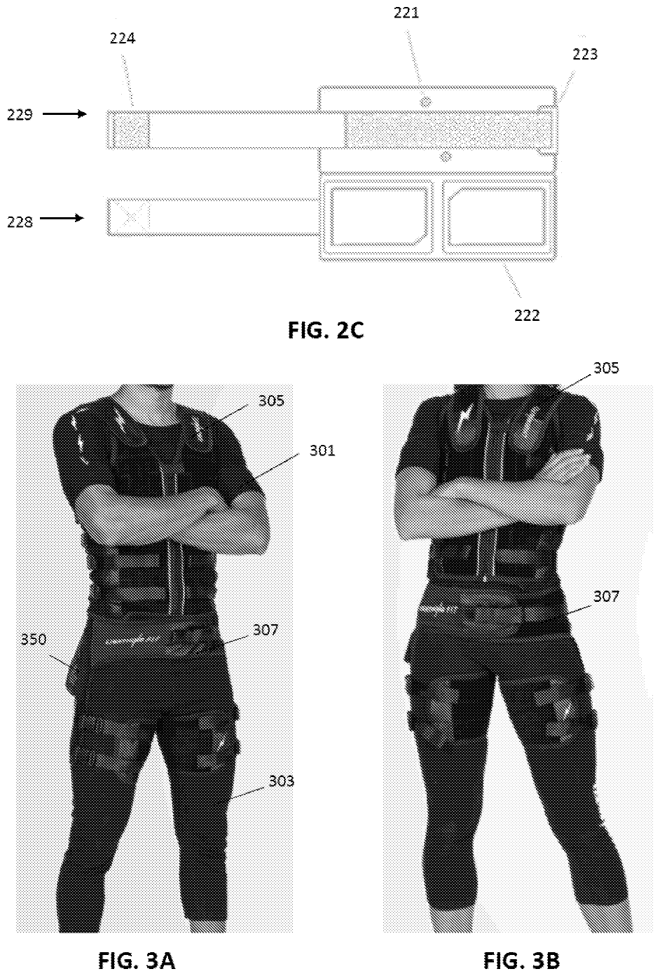

FIG. 2A shows an example of an upper torso portion 105. In this example, the upper torso portion includes a connector port 201 that may coupe with an integrated controller/power supply (not shown). The apparatus also includes a plurality of mechanical connectors (e.g., clasps, snaps, etc.) 202, and a plurality of adjustable straps (e.g., Velcro straps) 203, including side straps 204. The upper torso portion in this example also includes a zipper 205. A plurality of electrode pads 206 may also be integrated into the upper torso portion. The apparatus may also include connectors (e.g., buckles) 207 for coupling to a lower portion. Any of these connectors and/or straps may be adjustable and may include retainers 208.

FIG. 2B shows an example of a lower portion 107. The lower portion may also include one or more mechanical and/or electrical connectors 216 for coupling to an upper portion (onto which a power supply/controller may be attached). Alternatively or additionally the lower portion may hold the controller/power supply (and may provide power and control operation of the upper portion(s)). For example, the lower portion may include a pocket and/or attachment site 213 for the controller/power supply and/or may include a connector port 211. The lower portion may include a hip belt 212 for securing the apparatus to waist (e.g., onto or over an under suit). In some examples the apparatus may include adjustable straps and/or buckles and/or other components to adjust the fit 216. The lower portion may also include one or more electrode assemblies (including electrical pads 215) for applying EMS to a muscle/muscle group on the lower body.

FIG. 2C illustrates an example of an arm electrode support 119 shown from the top 229 and bottom 228 (user-contacting side). The arm electrode support may include one or more attachments (e.g., straps 221) including loops 223 and/or Velcro attachment portions 224. The arm electrode support may also include one or more electrode assemblies 222.

FIGS. 3A and 3B illustrate examples of EMS suits as described herein, worn on a user. In FIG. 3A the EMS suit including all of the components described above, including an upper portion 305, a lower portion 307. The upper and lower portions are coupled together, and a power supply/controller 350 is shown coupled to the lower portion. The user is also wearing an upper 301 and lower 303 under suit.

The EMS suits shown may include electrodes on the legs, e.g., quadriceps, buttocks, lumbar region, back, trapezious, and one or more on the abdomen, pectorals, and arms. The positions may be adjustable, within a predetermined or arbitrary range. For example, on the legs, the lower portion may be configured to allow adjustment of the position(s) of the electrodes in one or two positions, such as over the medial thigh muscle or the medial and lateral muscles. Electrodes may be positioned between 5 cm and 10 cm apart. In another example, the apparatus may be configured to allow adjustment of the position of the muscles of the abdomen, including adjusting the pairs of electrodes to be further or closer apart. The central abdomen may be adjusted and/or a more laterally separated position may be used.

FIGS. 4A-4B illustrate another example of an EMS suit apparatus similar to that described above, in which an under suit may not be needed. In this example, the apparatuses include a wetsuit-like appearance, and may be formed, at least in part of an elastic material, such as a polymeric material (e.g., neoprene, etc.) that is breathable, and is configured to hold the electrodes (e.g., the electrode assemblies) in contact with the skin. For example, in FIGS. 4A and 4B, the EMS suit may include an upper 405 and a lower 407 portion, or may be a unitary suite (e.g., integrated upper and lower portion). The suite may be formed of an elastic fabric and includes a closing system 447 (e.g., zipper). The electrode assemblies may be integrated into the suit but may be configured to allow selection of one or more alternative positions, e.g., to allow the user to adjust the separation the anode and cathode electrodes. Thus, the electrodes may include an input (e.g., strap, selector, etc.) for allowing or locking internal movement of the electrode assembly position. As mentioned above, any of these electrode assemblies may be configured to allow fluid (e.g., saline) to be applied as part of the electrode assembly. In the example apparatus shown in FIGS. 4A and 4B the electrode may be coupled to the power supply/controller 450 by internal wires (not visible).

As mentioned above, any of these apparatuses may include electrode assemblies configured as wet or wettable electrodes. For example, these apparatuses may be configured so that fluid (e.g., water, saline, etc.) may be added to wet a skin-contacting surface of the electrode assembly. Electrical contact is essential to proper function and control of an EMS apparatus. The electrode assemblies may be wet prior to applying the apparatus and/or after applying the apparatus, and in particular the electrode assemblies including the fluid reservoir regions, e.g., a porous material (e.g., sponge and/or wettable hydrogel, etc.). FIG. 5 illustrates an example of the application of fluid (e.g., water) 567 via a spray bottle to wet electrodes 565 (and in particular, to wet the porous skin-contacting surface of the electrode) on the inside of the suit 563. In some examples the suit may include one or more ports into which a fluid (e.g., water, saline, etc.) may be added. The fluid may be a conductive fluid.

Any of the apparatuses described herein may include sensors, e.g., motion sensors, position sensors, etc. that may confirm the position and/or activity of the user. Sensors may be included with the one or more electrodes and/or may be included with the controller (or power supply and/or controller, including integrated power supply/controller). The sensor(s) such as an accelerometer, may be used to confirm that the user is performing a predetermined action/exercise (as described below) and may therefore coordinate the application of the EMS with the prescribed movement(s). The sensor(s) may also be used as a safety trigger, for example, stopping or pausing (or in some cases decreasing) the application of EMS based on the sensed motion and/or position.

In general, the suits described herein may be cleaned and maintained by the user. For example, the suits may be treated with an antibacterial solution and rinsed with water. An anti-odor product may be applied following each use, and/or after applying the antibacterial solution. The suit may be dried, e.g., by hanging it in a drying area. An air-drying system may be used to expedite drying. Heated or room-temperature air may be used to dry the suit. In general, the suit may be washed, e.g., by soaking in a soapy solution at low concentrations. The suit may be washed and/or rinsed in cold water to clean (including removing excess salts from the added fluid and/or sweat).

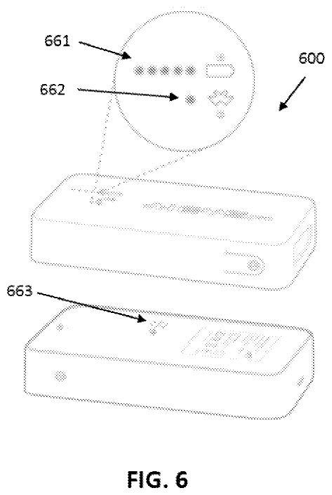

Also described herein are power sources and/or combined power sources (e.g., batteries) and controllers. FIG. 6 illustrates one example of a power source (e.g., battery) 600. This example may be used with an apparatus as shown herein and may include a simple user interface showing power level 661, wireless connectivity 662 (e.g., Bluetooth connection), etc. In some examples, described in greater detail below, additional user interface information (e.g., touchscreen) may be included. Any of these apparatuses may include an audio output 663 (e.g., speaker) that may be used as an output. The power source/controller may be, e.g., 500 g or less (e.g., 450 g or less, 400 g or less, 350 g or less, 300 g or less, 250 g or less, etc.). The apparatus may be relatively small (e.g., 20×10×5 cm or less, 18×8×3 cm or less, 16.5×8×3 cm or less, etc.). The apparatus may also include an on/off button that may be manually or automatically controlled. Any of these power sources may be configured as batteries, such as lithium ion (Li-Ion) batteries. The power source may include a charging port (e.g., mini-USB port). In some examples the power source may also or additionally include a port for connecting to the EMS suit, and/or a cable connected to the EMS suit. As mentioned above, in some examples the power source (or power source/controller) may be configured to be secured within a pocket in the suite and may electrically couple to the suit while within the pocket.

Any of these power sources and power source/controllers may include one or more emergency shutoff controls, or an override shutoff control. The shutoff control may be configured to immediately stop the application of power to the electrodes. In some configuration the shutoff control may be configured to completely shut off the apparatus; in other examples, the shutoff control may continue sensing/monitoring and processor functions but may disable the application of power to any of the electrodes (e.g., for delivery of EMS) until a rest condition is satisfied. For example, in some examples an emergency shutoff control (or an override shutoff control) may be included on the outer surface of the battery or battery/controller. In some examples the suit may have an integrated shutoff control on the front outer surface of the suit that may be easily actuated by the user.

Any of these suits may include one or more sensors (e.g., physiological sensors), including heart rate sensors, pulse oxygenation sensors, respiratory sensors, etc. In some examples the apparatus may be configured to trigger a safety shutoff of EMS if the sensors detect user physiological signals that exceed a predetermined safety threshold. For example, if the heart rate exceeds, e.g., 180 bpm (e.g., 155 bpm, 160 bpm, 165 bp, 170 bpm, 175 bpm, 180 bpm, 190 bpm, 195 bpm, 200 bpm, 205 bpm, etc.), and/or if the blood pressure exceeds a predetermine range, etc.

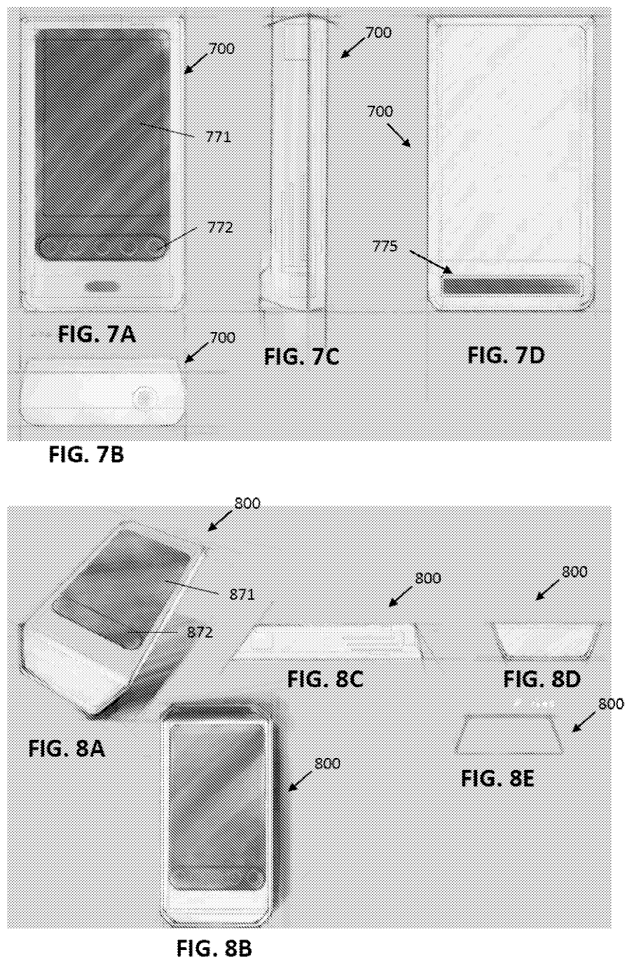

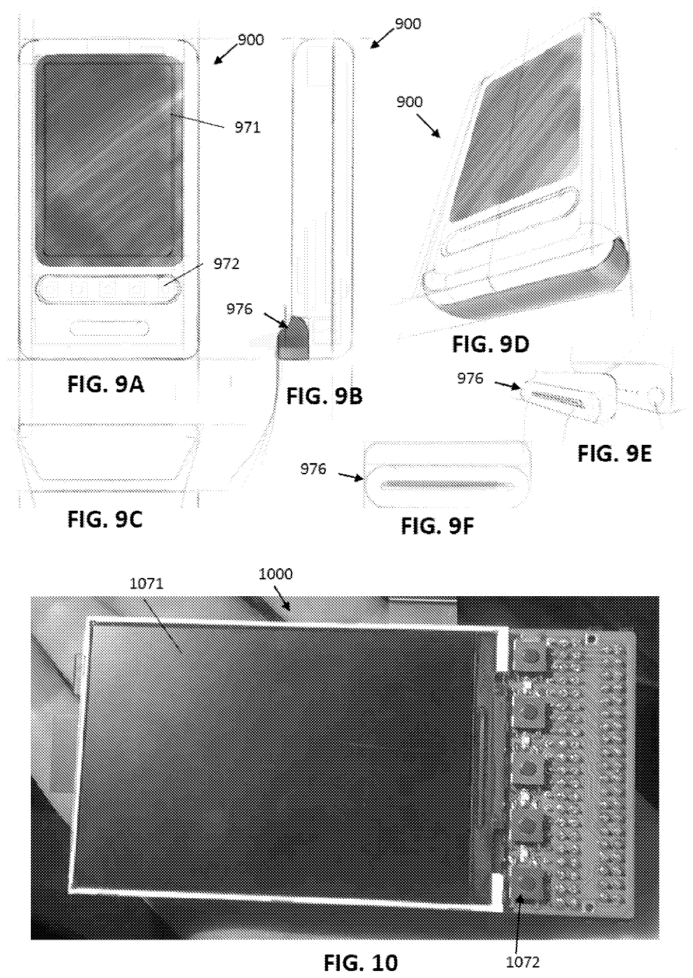

FIGS. 7A-7D illustrate one example of a combined power source/controller 700. In this example the combined power source/controller including a touchscreen input 771, and may include one or more additional inputs 772, including an emergency shutoff control. The combined power source/controller may also include an attachment (input) 775 for coupling to a charging source and/or for coupling to an input/output (including a cable input/output) on the EMS suit. FIGS. 8A-8E show another example of a combined power source/controller 800 similar to that shown in FIGS. 7A-7D, also including a display screen (which may optionally be a touchscreen) 871 and one or more inputs 872, including, e.g., an emergency shutoff control. FIGS. 9A-9F shown another example of a combined power source/controller including a display 971 and inputs 972. This example also shows nan interface or adapter 976 for coupling to the EMS suit and/or a charger for charging the power supply integrated with the controller.

FIG. 10 shows an image of an example of a combined power source/controller 1000, with the outer housing removed, showing the display screen 1071 and inputs 1072 visible. The controller may include one or more processors, memory, timer(s), and control circuitry, including wireless circuitry and/or power control circuitry.

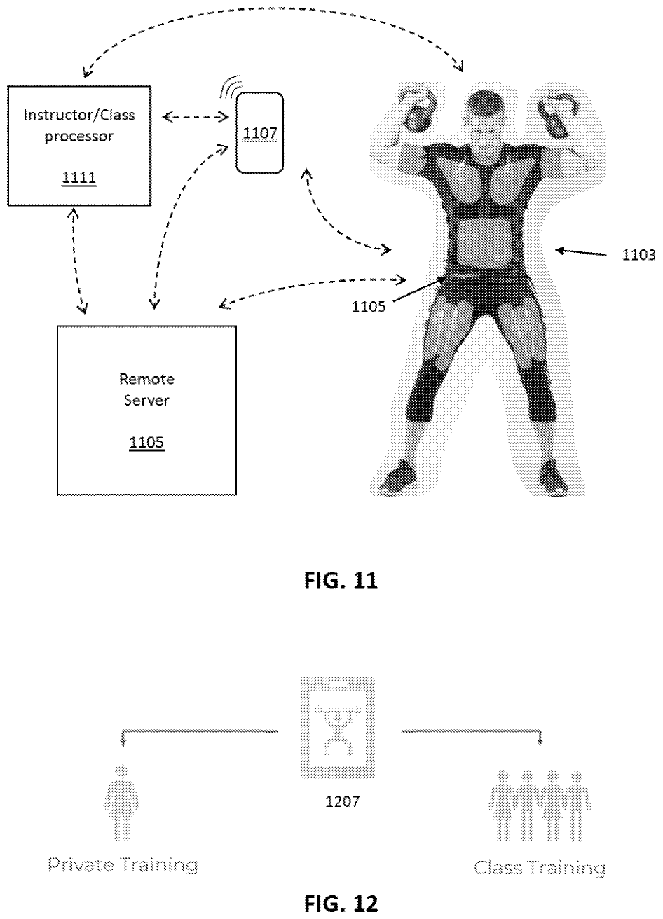

FIG. 11 illustrates one example of an EMS apparatus, including an EMS suit 1103, including a controller/power source that may operate with software on one or more of a user device (e.g., smartphone 1107), remote server 1105 and/or an instructor (or class) processor 1111. In FIG. 11, the EMS suit 1103 may be controlled by a worn controller 1105. The locally worn (EMS suit) controller may include the safety override control and may generally control the application of EMS to the electrodes/electrode assemblies in the suit, as described above. The locally worn controller (e.g., an integrated power supply/controller 1105) may communicate wirelessly, e.g., via Bluetooth (or other wireless technique) to any of the user device 1107, instructor/class processor 1111 and/or remote server. For example, the suit, which may be identified by a unique identifier associated with the user (e.g., name, number, address, etc.) may receive instructions for delivering a predetermined EMS protocol corresponding to a desired training regimen. The protocol may be delivered by the local controller but may be run in combination with the remote server, instructor class/server (for example, for group exercise) or from the user device (e.g., smartphone 1111).

In particular, the apparatus may be configured so that during a training episode, a selected or prescribed training regimen may be provided to the user, instructing the user to perform one or more actions. As mentioned above, a sensor, e.g., a motion sensor (e.g., accelerometer) may be included as part of a combined power source/controller and the controller may confirm that the user has begun, is in the midst of continuing to perform, or has completed, a prescribed movement before applying or continuing the application of EMS.

In general, the application of EMS may be targeted to a particular set of muscles or muscle groups corresponding to a particular activity. For example, the apparatus may be configured to apply a workout targeting a particular user goal, such as increasing endurance, mobility, and/or strength. These workouts may include a defined set of movement or actions (e.g., exercises, yoga/stretching poses or movements, etc.) and may be presented to the user concurrently with the application of EMS to one or more muscles (or muscle groups) related to the movements or actions.

For example, strength training routines may include resistance exercises (weights, bands, bodyweight, etc.), core strength, high-intensity interval training, etc., and may target specific muscles. The apparatus may automatically apply EMS in a coordinated manner with the presentation (and presumed performance) of the movements and/or may detect the user's movements and apply EMS when the user is performing the appropriate corresponding movement or shortly thereafter. In some examples the apparatus may therefore provide immediate feedback to the user that the movement is being performed within a desired level of activity, further reinforcing the effects of the EMS.

In any of these apparatuses, the intensity of the apparatus may be automatically adjusted to either adjust the applied EMS or to set the range of EMS intensities within which the user may select intensities (e.g., high, medium, low, off, or X % of 100%, where the range of 100% is set automatically by the system).

In particular, the apparatuses described herein may control and/or set the maximum EMS power/intensity that may be applied to a particular user based at least in part on one or more of: (1) an initial baseline (e.g., starting baseline) for the user; (2) the user-provided input(s); and (3) the historical (including within the last z hours or days) application of EMS by the same user. In any of these example the baseline power may be determined for all of the electors (a collective baseline power) or for individual and/or subsets of electrodes. In any of these examples a baseline power module may determine the baseline power for the user. Thus, the apparatus may control the applied EMS power/intensity specific to each muscle or muscle group (e.g., the maximum intensity/power applied to the quadriceps may be different from the maximum power/intensity applied to the biceps, for example). Alternatively, the maximum power/intensity of the EMS applied may be the same across all muscles/muscle groups. The apparatuses described herein may adjust the EMS power/intensity by adjusting one or more of the pulse frequency applied (e.g., between 0/off and 120 Hz, e.g., between 0-100 Hz, between 50-120 Hz, between 60-120 Hz, between 70-120 Hz, between 80-100 Hz, between 70-100 Hz, etc.), the current applied (e.g., between about 0.2-120 milliamps (mA), between about 1-90 mA, between about 1-100 mA, between about 5-110 mA, between about 1-120 mA, etc. or any range within these), the pulse width (e.g., between about 100 microseconds (μs) to about 500 μs, between about 200 us to about 450 μs, between about 150 us to about 400 μs, or any ranges therebetween). In some examples the EMS power/intensity may be modulated by adjusting the ramp-up time (time to ramp up to a maximum applied current) and/or by adjusting the frequency within an application session (increasing or decreasing the frequency).

In general, the apparatuses described herein may be configured to apply power to just a subset of the electrodes (e.g., muscles or muscle groups) and may apply power to separate sets of electrodes corresponding to different muscles in an alternating manner, to avoid concurrent stimulation of multiple sets of electrodes.

The apparatuses described herein may be configured to safely apply EMS by controlling the applied energy (e.g., frequency and/or current and/or pulse width and/or ramp-up/ramp down time) based on a combination of on one or more of: (1) an initial baseline (e.g., starting baseline) for the user; (2) the user-provided input(s); and (3) the historical (including within the last z hours or days) application of EMS by the same user. In particular the apparatuses described herein may automatically set the maximum applied EMS power and/or intensity by estimating a maximum specific to a particular user base on the user's specific baseline and the user's recent (e.g., within the last z hours, where z is between about 8 hours or less, about 24 hours or less, about 36 hours or less, about 48 hours or less, about 60 hours or less, about 72 hours or less, etc.).

An initial baseline may be set based on a user's initial response to questions, such as the user's age, fitness level, general or specific health concerns, etc. In some examples the apparatus may perform an automated question and answer/testing session to set a baseline/initial user level. For example, the user may respond to questions regarding their ability to feel certain EMS inputs one or more (or all) of the electrodes. The user may also be asked to perform various actions (e.g., exercises) while wearing the apparatus and/or without wearing the apparatus and may provide feedback by self-reporting or via one or more sensors (e.g., heart rate, pulse oxygenation, accelerometer/position sensors, etc.).

The baseline data may be estimated, and an initial maximum level of EMS intensity/power may be determined. The initial baseline may be determined from population data of similarly-situated users (e.g., by age group, gender, health, weight, height, etc.). Initial baseline data may be set in part based on user-reported response to a variety of different EMS stimulation levels for each (or a subset of) muscles/muscle groups.

A dataset for each user may be maintained locally (e.g., on the user-specific EMS suit/controller) and/or may be kept in a remote database and accessed by the user-specific EMS. The user information (data) may include specific responses to the initial baseline data collection and/or the initial baseline values estimated by the system, including initial baseline values specific to each muscle or a subset of muscles). In some cases, initial baseline values for different muscles may be determined based on a patient-specific estimate for one or more muscles (e.g., quadriceps, biceps, pectorals, etc.).

User-specific baseline data may be adjusted periodically. User-specific data may be secured. For example, if user-specific date is recorded in a remote database it may be anonymous and indexed by a separately secured key corresponding to a particular user. In some cases, baseline information may be specific to an EMS system (e.g., EMS suit) and/or specific to a user. A user may be uniquely associated with a particular EMS suit).

The maximum EMS intensity/power that may be applied to the user may be adjusted with operation of the system. In general, the initial (e.g., baseline) EMS intensity/power may be set low, to avoid harming the user. With consistent use, the maximum intensity may be adjusted. For example, the apparatus may be configured to increase the maximum intensity with regular use and/or with user-requested increase.

As a safety, any of the apparatuses described herein may reduce and/or reset the maximum if EMS is not applied within a particular timeframe (e.g., if it has been more than x hours or days since the last EMS use). For example, for every x hours that the user has not operated the EMS apparatus the maximum intensity/power may be scaled towards the initial baseline (e.g., if no EMS has been performed by the user within 4 days, 5 days, 6 days, 7 days, 8 days, 9 days, 10 days, 11 days, 12 days, 13 days, 14 days, etc.).

Further, the system may lock out or prevent EMS from being applied more frequently than a predetermined time period. For example, the apparatus may be configured to prevent the user from applying EMS more than one session (or more than a maximum number of minutes or a maximum aggregate energy) every x hours or day (e.g., no more than once per 20 hours, per 24 hours, per 30 hours, per 36 hours, per 40 hours, per 44 hours, per 48 hours, per 52 hours, per 56 hours, per 60 hours, etc.).

Any of the apparatuses described herein may gradually increase the intensity of the EMS (e.g., the power of the EMS), e.g., by increasing one or more of the frequency, pulse width, amplitude, etc., over the course of a treatment session, up to a limit of the maximum intensity. For example, the apparatus may automatically increase the applied EMS power from an initial starting level up to 100% of the maximum EMS power/intensity available to the patient as calculated for a particular session (based on the baseline, historical, and/or user-selected input). In one example, a new user may have a maximum baseline power for their first session of 60 Hz, and a pulse width of about 200 us and an amplitude (which may depend on the electrode parameters) of, e.g., 100 mA. The applied intensity/power may be a function of one or more of these parameters. The apparatus may increase the applied EMS power/intensity from 50% of this value up to the maximum 100% (e.g., starting at 30 Hz and increasing to 60 Hz, etc.), which may be specific to a particular muscle or set of muscles.

In general, these apparatuses may include software that may perform any of these methods. For example, the software may be an application software (app) that is configured to run on the user's device (e.g., smartphone) and/or on the power supply/controller. The software may be configured to control the EMS to a particular use or to coordinate the control of a group of users that may be exercising together, e.g., as part of a class. For example, FIG. 12 schematically illustrates a user software 1207 that may be used to assist the user in private training or in training a part of a class.

The application software 1207 may allow a user to select a particular training session and/or class and may coordinate the application of the training session and the application of the EMS. In some examples the application software may record the progress and may adjust the maximum possible EMS intensity/power for each session, as described above. The application software may therefore automatically adjust the settings (EMS settings) for each workout.

The sessions (“workouts’) may start the EMS with settings based on the individual base settings (baseline) as mentioned above, and the apparatus may optimize the maximum possible EMS intensity and/or power for each setting, as well as the onset of this maximum during the course of a session. As described above, this may therefore be customized to each user and specific to their body and fitness level and configured to prevent harm or discomfort to the user. The apparatus may recalibrate the base settings.

Any of these apparatuses may include a user interface that may be displayed, for example, on the power supply/controller and/or on the user apparatus (e.g., smartphone, television/display, etc.). This user interface may include input/outputs for each of the EMS electrodes and/or corresponding muscle groups, including showing an intensity level (e.g., as frequency, amplitude, pulse width, and/or some combination or derived value of these, e.g., generically “intensity”). The user may be permitted to select a value for each or some of these which may be limited to the maximum level selected or set by the system. The user interface may also show the corresponding positions/movements/exercise and/or may display an instructor or model performing or guiding the user in performing them. In general, the apparatus (e.g., software, firmware, etc.) may coordinate the application of EMS via the EMS suit to the session being performed, including coordinating activating of the appropriate electrodes.

The techniques and systems described herein may also allow for one or more devices to conserve resources with respect to communications bandwidth resources, processing resources, memory resources, power resources, and/or other resources, as described herein. Additional technical effects can also be realized from the implementation of the technologies disclosed herein. Described herein are example processes, as well as systems and devices comprising one or more processors and one or more memories, as well as non-transitory computer-readable media storing computer-executable instructions that, when executed, by one or more processors perform various acts and/or processes disclosed herein.

In general, the methods and apparatuses described herein may include or operate with a machine learning agent (e.g., a trained neural network). In general, the methods and operations described above may be performed with a trained neural network. Any of these methods and apparatuses may operate with a data store (e.g., database) that may be used to store and provide (be retrieved from) data related to predefined patterns of movement (e.g., “exercises”), including variations and patterns of settings (or setting adjustment relative to the base setting). The database (data stores) may also include session data from sessions with any of the apparatuses described herein, which may include: programmed exercises selected for and/or used during the session, and prescribed adjustments made to the settings during the performance of the exercise(s), manual adjustments made by the trainer during the session to the settings. In general, these database may include any of the data generated during the operation of the apparatus (e.g., during a workout) from any of the sensors described herein (e.g., accelerometers, positions sensors, force sensors, etc.). This data may be annotated by the user, by a therapist/coach/trainer, etc. The data may also include settings applied and any changes in the settings, as well as comments (or recommendations) from the user and/or therapist/coach/trainer, etc. The database may also include data specific to the user, and in particular data that does not uniquely identify the user (e.g., weight, height, fitness state, goals, conditions/impairments, etc.). In some examples the database may include user profile information. In some cases the database may include user or institute affiliation and/or subscription information. In some examples the database may include progress over time, towards goals and other measures (e.g., SMM, Body Fat, Weight, etc.). The annotated data from users and/or therapist/coach/trainer (or in any of these examples, physicians) may include feedback from the trainers pre- and/or post-workout. In some examples the database may include data from additional worn sensors (e.g., wearables, such as but not limited to pedometers, smartwatches, ECG data, etc.); these sensors may be redundant with sensors included in the apparatus, e.g., garment. For example, the database may include hear rate data from the garment and/or form a wearable. In some examples this data may be condensed, such as averaged, filtered, summarized, etc. For example the data in the database may include maximum and/or minimum values for any of these parameters, such as but not limited to heart rate. In any of these methods and apparatuses, the database may include a resting parameter value for any of these parameters (e.g., resting heart rate).

The database, or a portion of the database (e.g., a subset) may be used and/or designated as a training database for training the machine learning agent. In some examples the machine learning agent or agents may be trained to identify what sequential movements (e.g., exercises) and/or workouts a user is performing, and may control, or may provide input to the one or more controllers to control, the output of the EMS electrodes based on the determined series of movements, including which electrodes to make active, and/or which levels of energy to apply. As described above, the controller (with or without the assistance of the machine learning agent) may set and/or adjust, e.g., determine, the base level of energy applied overall and/or for one or more specific electrodes or set(s) of electrodes. In any of these examples the machine learning agent may recommended the settings or adjustments to the settings; these recommendations may be made to the user and/or a therapist/coach/trainer. Alternatively or additionally the controller may automatically or semi-automatically (e.g., with approval of the user and/or therapist/coach/trainer) adjust or set the parameters applied by the electrodes, including which electrodes to operate based on the machine learning agent.

In some examples a movement identification module may be provided to identify the type of exercise based on the sensor data from the EMS apparatus. The movement identification module may be determined without the use of a machine learning agent, e.g., by applying a statistical model, a look-up table, etc. In some examples the movement identification module may include a machine learning agent. In general, any of the methods and apparatuses described herein may include a movement identification module to determine (e.g., output) the type of movement (e.g., exercise) being performed. The movement identification module may be engaged periodically or in an ongoing matter, and may respond relatively quickly, e.g., within 2 min or less, 90 seconds or less, 60 seconds or less, 45 seconds or less, 30 seconds or less, etc.) to identify a movement being performed. For example, the movement identification module may examine a window of time immediately prior (e.g., based on sensor output over this time period) to identify a movement being performed (e.g., exercise) and to output the result when the confidence level is above a threshold (e.g., indicating a particular type of compound movements or exercises). The output may be presented to the controller to automatically or semi-automatically adjust the electrodes of the EMS apparatus (e.g., the settings and/or which subset of electrodes are used).

In any of these examples the apparatus or method may include an electrode energy setting module that may determine the settings to be applied to all or some of the electrodes. The electrode energy setting module may include a trained machine learning agent, which may receive input from the movement identification module or a manual input of the type of movement (e.g., exercise) being performed, and/or may receive input from the sensor(s) and/or the user and/or the therapist/coach/trainer, and may output EMS settings appropriate to the indicated or determined set of movements. In some cases the electrode settings module may alternatively or additionally adjust the baseline EMS power for a user as described above (e.g., the baseline power module).

The baseline power module may include a trained machine learning agent for determining the baseline power for a particular user. In general, the baseline power module may select the baseline power setting(s) as described above, including recent use/nonuse of the system by the user; this data may be included in a database accessible to the apparatus, including in particular to the controller and/or baseline power module.

In general, the apparatuses described herein may include one or more trained machine learning agents that may be part of one or more modules and/or one or more controllers, including remote and/or local controller(s). In some examples a trained machine learning agent (e.g., neural network) may be trained based on a supervised, semi-supervised, or unsupervised technique. In any of these examples the machine learning agent may be trained using the database described above, which may include types of movements (including exercises), sensor information (location, sensor type, values), user data (e.g., exercise history, age, gender, body weight, height, body mass index (BMI), general health, etc.), electrode settings/changes in electrode settings, etc. In some examples the trained machine learning agent may be trained to identify the type of movements (e.g., exercise) being performed, including which muscles or muscle groups are being trained and/or the electrode sets/subsets and/or values used.

Modules described herein may be part of the controller or controllers and may be local (e.g., wired/coupled directly) to the EMS apparatus or remote (e.g., coupled via a wireless connection and/or via a network). In some examples the EMS apparatus may include a local controller that communicates either directly or indirectly (e.g., via a user's smartphone, local tablet, laptop, etc.) and/or wirelessly, e.g., Bluetooth, etc., to a remote server.

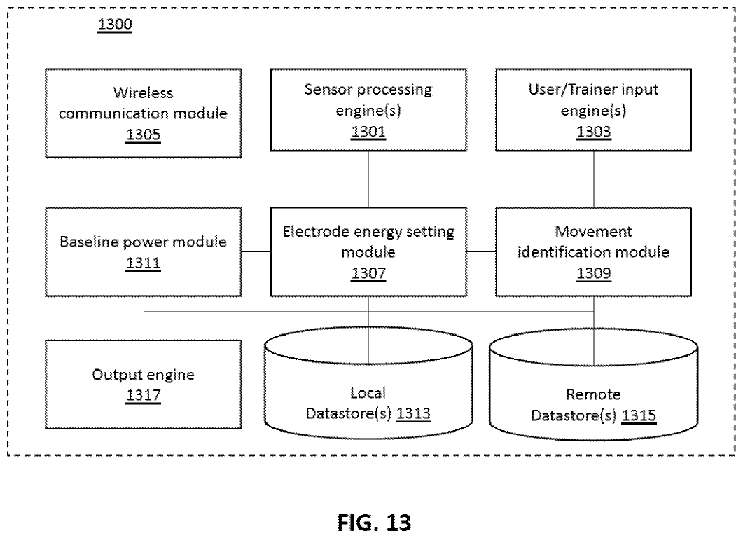

FIG. 13 schematically illustrates one example of a control subsystem (e.g., controller or controllers) for an EMS apparatus as described herein. In this example the control subsystem 1300 may be used with one or more electrodes distributed across one or more garments such as a top (e.g., shirt, vest, band, belt) and bottom (e.g., shorts, pants, etc.). The control system shown may include all or some of these engines and modules, in addition to a memory and one or more processors forming the substrate for these engines and modules. Some or all of these modules and engines may be local, and some or all of these modules and engines may be remote. For example, these modules or engines may wirelessly communicate (via the wireless communication module) with each other and/or with the remote 1315 and local 1313 datastore(s). In the example schematic shown in FIG. 13, the control subsystem includes one or more sensor processing engine 1301 that may receive and process (pre-process) data from the one or more sensors on the EMS apparatus, including accelerometers, force sensors, pressure sensors, etc. The sensor processing engine may interrogate the sensor(s), process the sensor data (e.g., filter, amplify, average, etc.) and may store and/or transmit the sensor (or processed sensor) data. The control subsystem may also include a user and/or therapist/coach/trainer input engine 1303 that may receive and/or process input from the user and/or a therapist/coach/trainer. Input may be in response to a user interface as described above (e.g. a prompt or input on the apparatus or from a device in communication with the apparatus). User/Trainer input engine may store/transmit and/or process the input from the user and/or therapist/coach/trainer. In FIG. 13 the control sub-system 1300 may also include one or more baseline power module 1311 as discussed above, which may determine and/or set baseline power for all or some of the electrodes. The control subsystem may also include one or more movement identification modules 1309, as described above that identifies the movement(s) including exercises being performed, and one or more electrode energy setting modules 1307, also discussed above. Finally, a control subsystem may include an output engine for providing output to control the electrodes (e.g., applying power to the one or more electrodes as determined herein), selecting which electrodes or sub-set of electrodes to activate (apply power to) and/or output to display and/or store.

The control subsystem may also include power regulator engines (including battery charging circuitry and logic) and one or more signal generator regulator engines, for communicating with or controlling a signal generator for applying power to the selected EMS electrodes in the apparatus.

As understood herein, computer system can be implemented as an engine, as part of an engine or through multiple engines, and/or as part of a module, portion of a module or through multiple modules. An engine or module may include one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors or a first engine and a second engine can share one or more processors with one another or other engines. Depending upon implementation-specific or other considerations, an engine or module can be centralized or its functionality distributed. An engine or module can include hardware, firmware, or software embodied in a computer-readable medium for execution by the processor. The processor transforms data into new data using implemented data structures and methods, such as is described with reference to the figures herein.

The engines and modules described herein, or the engines and modules through which the systems and devices described herein can be cloud-based engines or modules. As used herein, a cloud-based engine or module is an engine or module that can run applications and/or functionalities using a cloud-based computing system. All or portions of the applications and/or functionalities can be distributed across multiple computing devices, and need not be restricted to only one computing device. In some embodiments, the cloud-based engines or modules can execute functionalities and/or modules that end users access through a web browser or container application without having the functionalities and/or modules installed locally on the end-users' computing devices.

As used herein, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein, can be cloud-based datastores. A cloud-based datastore is a datastore that is compatible with cloud-based computing systems and engines.

In general, electrodes may be referenced based on the nearest muscle type/group (e.g., right/left quad, right/left hamstring, right/left glute, lumbar, right/left lats, traps, abs, chest, right/left bicep, right/left forearm, etc.). The controller (or control subsystem) may also determine and/or confirm the presence of any of these electrodes and/or may confirm sufficient contact (based on an electrical property of the electrode, such as bioimpedance) with the user's skin.

A trained machine-learning agent may be used as part of any of the apparatuses and methods described herein. In particular, a trained machine-learning agent may be used to determine what settings (e.g., electrode settings) including what sub-set of electrodes, what power, duration and/or intensity, to be applied to each of the electrodes of the EMS apparatus.

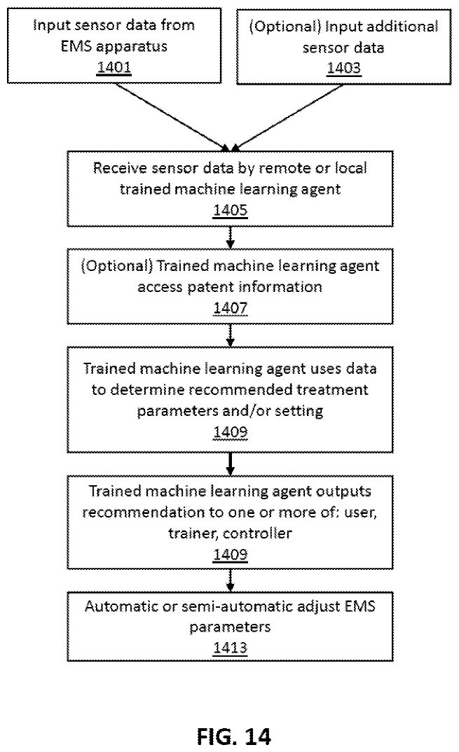

For example, FIG. 14 illustrates a schematic example of the use of a machine-learning agent to propose and/or set EMS parameters. In this example the control sub-system may receive input from one or more sensors, as described above 1401 and this input may be passed to the trained machine learning network (e.g., a movement identification module and/or an electrode energy setting module) 1405 that may include or access the same or different machine learning agent(s). Optionally the method may include receiving input from sensors worn or associated with the user, such as wearable electronics (e.g., heart rate monitors, blood glucose monitor, etc.) or the like 1403. Additionally, in some examples the control subsystem, and the trained machine learning agent associated therewith, may receive input from the user and/or therapist/coach/trainer (not shown) and/or may access information specific to the patient 1407. In some examples, the trained machine learning agent may receive at least input from all or some of the sensors 1401 and the exercise being performed (or intended to be performed. In some cases the same trained machine learning agent may determine the exercise being performed; in some examples the machine learning agent may receive as input the exercise to be performed, e.g., from a movement identification module.

The trained machine learning agent may then output recommendations for the settings (or adjustments to the settings) and/or modifications of the baseline power to be applied 1409. This output may be made to the user (e.g., via an output such as a display, indicator, etc.), the therapist/coach/trainer, etc., and/or to the control subsystem itself, that may automatically or semi-automatically (based on confirmation or other input from the user and/or therapist/coach/trainer) adjust the parameters as determined.

In one example the apparatus includes a control sub-system with a trained machine learning agent that is configured as an electrode energy setting module; the control sub-system may also include a movement identification module with the same or a different trained neural network. In some examples the electrode energy setting module and the movement identification module may be part of the same module and include the same trained neural network (generically referred to as an electrode energy setting module). The control sub-system may receive data input from a plurality of different motion sensors (e.g., accelerometers) positioned on the EMS suit, e.g. in the arms, torso, legs, etc.) and may process this information to determine, over a window of time (e.g., 2 seconds, 3 seconds, 4 seconds, 5 seconds, 6 seconds, etc.) that the user is performing a set of compound movements, e.g., moving the quadriceps/legs in a regular manner indicating that the person is performing leg exercises generally, and in some examples, squats specifically. This may be determined in real time. The electrode energy setting module may receive this information, and may receive additional data from one or more other sensors (e.g. heart rate sensors, skin conduction, respiratory sensors) and/or data specific to the patient (e.g., age, general health, prior workout(s), weight/BMI, etc.) and may adjust propose settings to the one or more electrodes of the EMS suit. For example the settings for non-relevant muscle group electrodes (lumbar, lats, traps, abs, chest, arms) may be set to 0 (“false”) so that power is not applied by these electrodes, whereas power may be applied to the electrodes associated with the quads, hamstrings, and glutes. In some examples the control sub-system may also confirm appropriate electrical contact (e.g., by bioimpedance detection) with the electrodes and the user. The electrode energy setting module may further determine what level to recommend power is applied (current/voltage applied, frequency, etc.) based on the trained machine learning agent output for the patient. The trained machine learning agent may be trained and/or may use the baseline level described above, and adjust the output accordingly. In some examples the trained machine learning agent recommendation may be based on similar patients (e.g., height, weight, physical fitness level, etc.) having successful outcomes as indicated by the training dataset. Thus, the machine learning agent may customize the training regimen, including in particular the EMS power applied to the patient.

In general, these apparatuses may automatically turn on/off the appropriate sub-set of EMS electrodes relevant to a particular set of compound movements. As just described, shortly after beginning the movement, in real time (e.g., with about 1-10 seconds), the apparatus may detect what the exercise is and may adjust/recommend the appropriate settings, which may be specific to the user. The EMS apparatus may therefore detect movement of the user wearing the apparatus (e.g., changing the plane/position of the body, etc.).

### Compliance

Any of the apparatuses and methods described herein may be configured to determine and/or indicate compliance. For example, these apparatuses may include a control subsystem with a compliance monitoring module that determines when (or that) a user is operating the EMS apparatus, or that is operating the EMS apparatus according to a generic or user-specific schedule. The control subsystem may output information to the user, to a therapist/coach/trainer and/or to a physician, database, etc. The compliance data may be provided as a schedule of use/non-use (e.g., used X times in Y days), a list of specific dates used and/or not-used, an indicator of the percentage or duration of time used, etc. In some examples the compliance monitoring module may include one or more reporting thresholds. For example, the compliance threshold may be failure to use the apparatus less than some number of minutes/hours per week, etc. The compliance threshold may be adjustable (e.g., by user, by a physician, trainer, coach, clinician, insurance provider, etc.). In some examples the compliance monitoring module may report use.

The compliance monitoring module may be configured to determine when use of the apparatus constitutes sufficient use. For example, the compliance monitoring module may detect the start of use (e.g., applying energy to the apparatus) and/or the duration of use (e.g., period of time energy is applied), or the total and/or average energy applied during a session, or per day, three-day period, week, 10 day period, two-week period, month, etc.

In any of these examples the compliance monitoring module may confirm or indicate that the apparatus is being worn by a specified user. User identity may be confirmed manually (e.g., by the user entering his/her/their name or identifying code) and/or automatically, including examining one or more biometrics based on the sensors. For example, the compliance monitoring module may be determined based on the characteristic gait, as determined by motion sensors on the apparatus.

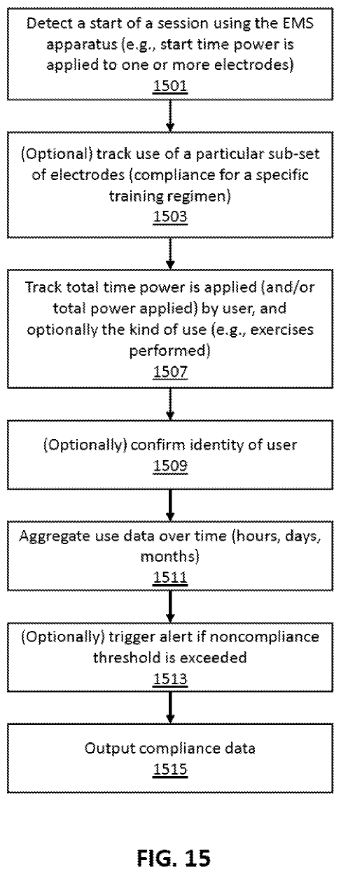

FIG. 15 illustrates one example of a method of determining compliance. As mentioned the apparatus may include a compliance monitoring module to perform this method. In general, the method may include monitoring the use of the EMS apparatus specific to a user to track the use over time of the EMS apparatus for that user. For example, the method may include determining that a user has started an EMS session, e.g., by recording the time that power is first applied to one or more electrodes 1501. Optionally, compliance may be specific to a particular use of the EMS apparatus (e.g., to train the legs, arms, etc.) and therefore the apparatus may specifically track power applied to a subset of the electrodes (instead or in addition to tracking all use) 1503. The delivery of power may be confirmed by the use of one or more sensors, e.g., bioimpedance sensors, confirming that the EMS apparatus is properly worn and/or that energy was delivered to the user. Thus, the compliance monitoring module may include input from one or more impedance sensors to confirm delivery of energy to the user.

The apparatus may determine the duration of use, e.g., the duration in which power is applied to the apparatus 1507. This may be tracked as time (e.g., seconds, min, etc.) and/or as the power applied during a session. The use, corresponding to the period during which power is applied to the user, may be tracked and/or aggregated 1511 for a compliance period. The compliance period (days, weeks, months, etc.) may be set by the user, therapist/coach/trainer, physician, insurer, clinic/hospital, etc. including third-party owners of these apparatuses (teams, companies, gyms, etc.).

As mentioned above, in some examples if the use over the compliance period, and in particular if the verified use (e.g., based on the bioimpedance sensor data) is less than a compliance threshold value, the apparatus may trigger an alert, which may be output to the user and/or a third party (e.g., therapist/coach/trainer, etc.) 1513. In some cases the user and/or third party may be sent an alert if the use is approaching the compliance threshold. At any appropriate time, based on a schedule or upon receipt of an inquiry, the apparatus may output compliance data (e.g., user, including verified use, over time) 1515.

As mentioned, optionally in any of these apparatuses and methods, the EMS apparatus may confirm the identity of the user, either manually and/or automatically 1509. This may be done before the start and/or during and/or immediately after a use session.

### Applications: RLS and Physiotherapy

Any of the methods and apparatuses described herein may be used for one or more therapeutic indication, such as for physiotherapy by a patient (user) recovering from an injury, surgery, etc. Examples of these indications include, but are not limited to neuromuscular disorders (Parkinson's, etc.), obesity, pre- and/or post-surgery, hypertension, etc.

For example, described herein are methods and apparatuses for treating Restless Leg Syndrome (RLS). Restless Legs Syndrome or Willis-Ekbom Disease, is a neurosensory disorder, can be treated with pharmaceuticals or conservatively. This methods and apparatuses described herein may be used in particular to treat RLS instead of, or in addition to, other techniques for treating RLS, including whole body vibration, pneumatic compression, and near-infrared light. For example, RLS may be treated by the application of EMS either to the lower trug (e.g., legs, buttocks, etc.) including the muscles of the lower legs (bilaterally or unilaterally). In some examples, RLS treatment by EMS may be applied to other muscles of the body as well, including the pectorals, back, etc.

In general, RLS may be treated by applying EMS during one or more treatment sessions as described above. Treatment may be passive (e.g., without the user/patient) exercising, or active, including actively moving or exercising the legs. One or more treatment may be applied, including one or more 5-40 minute sessions (e.g., 10-30 min, 10-20 min, etc.). Multiple treatment sessions may be applied. For example, a treatment session may be repeated as needed, or every x days (e.g., every 2 days, every 3 days, every 4 days, every 5 days, every 6 days, every 7 days, every 8 days, every 9 days, every 10 days, etc.). In some examples, treatment may be repeated a decreasing frequency after an initial number of treatments.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

In general, any of the methods and apparatuses described herein may be used for physiotherapy. For example, EMS may be used as a rehabilitation tool for a range of neuromuscular conditions in adults and children, including (but not limited to) stroke, spinal cord injury, ABI and cerebral palsy. EMS may be used alone or with other conventional physiotherapy adjuncts. For example EMS may be used to reverse muscle atrophy and improved muscle strength; for support of function (i.e. stepping of the foot during gait), for improved local circulation and reduction in skin breakdown; for increasing and/or maintenance joint range of motion, for reduction of spasticity or muscle spasms; for an increase in cardiovascular function (e.g., via simultaneous activity of large muscle groups); for habilitation (e.g., learning new activity via movement normally unobtainable); for maintenance of bone density; and/or for restorative therapy (e.g., CNS cell birth & CNS myelination).

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. An electrical muscle stimulation (EMS) suit apparatus, the apparatus comprising:
a garment comprising an elastic material that is configured to conform to a user's body;
a plurality of electrode assemblies on an inner face of the garment, the electrode assemblies each configured to sit flush against the user's skin;
a plurality of sensors distributed on, in, or over the garment;
a plurality of electrical connectors electrically coupling the electrode assemblies to controller for applying EMS; and
a controller configured to control the application of energy to individual electrodes assemblies of the plurality of electrode assemblies, wherein the controller is configured to receive sensor data from the plurality of sensors and to identify a type of exercise being performed by the user, and to set a subset of the plurality of electrode assemblies to activate based on the identified type of exercise.

2. The apparatus of claim 1, wherein the controller comprises a trained machine learning agent configured to identify the type of exercise being performed by the user.

3. The apparatus of claim 1, wherein the controller is further configured to recommend or adjust the energy applied by the subset of the plurality of electrode assemblies based on the type of exercise identified.

4. The apparatus of claim 3, wherein the controller comprises a trained machine learning agent configured to recommend or adjust the energy applied by the subset of the plurality of electrode assemblies and based on sensor data from the plurality of sensors.

5. The apparatus of claim 3, wherein the controller comprises a trained machine learning agent configured to recommend or adjust the energy applied by the subset of the plurality of electrode assemblies and based on user-specific data.

6. The apparatus of claim 1, wherein the user-specific data comprises one or more of: user past performance, user height, user weight, user BMI, user physical condition, user age, user biometric data from the plurality of sensors.

7. The apparatus of claim 1, wherein the garment comprises one or more of: a shirt, vest, pants, or shorts.

8. The apparatus of claim 1, wherein the plurality of sensors comprises one or more of: motion sensors, bioimpedance sensors, and position sensors.

9. An electrical muscle stimulation (EMS) suit apparatus, the apparatus comprising:
a garment comprising an elastic material that is configured to conform to a user's body;
a plurality of electrode assemblies on an inner face of the garment, the electrode assemblies each configured to sit flush against the user's skin;
a plurality of sensors distributed on, in, or over the garment;
a plurality of electrical connectors electrically coupling the electrode assemblies to controller for applying EMS; and
a controller configured to control the application of energy to individual electrodes assemblies of the plurality of electrode assemblies, wherein the controller comprises a trained machine learning agent configured to recommend and/or adjust the energy applied by the plurality of electrode assemblies based on data from the plurality of sensors.

10. The apparatus of claim 9, wherein the controller is configured to identify the type of exercise being performed by the user based on the plurality of sensors.

11. The apparatus of claim 10, wherein the trained machine learning agent is configured to recommend or adjust the energy applied by the subset of the plurality of electrode assemblies based on user-specific data and exercise type.

12. The apparatus of claim 9, wherein the controller is configured to automatically or semi-automatically adjust the energy applied by the plurality of electrode assemblies based on the recommendation of the trained machine learning agent.

13. The apparatus of claim 9, wherein the trained machine learning agent is configured to recommend or adjust the energy applied by the subset of the plurality of electrode assemblies based on data from the plurality of sensors and based on user-specific data.

14. The apparatus of claim 13, wherein the user-specific data comprises one or more of: user past performance, user height, user weight, user BMI, user physical condition, user age, user biometric data from the plurality of sensors.

15. The apparatus of claim 9, wherein the garment comprises one or more of: a shirt, vest, pants, or shorts.

16. The apparatus of claim 9, wherein the plurality of sensors comprises one or more of: motion sensors, bioimpedance sensors, and position sensors.

17. An electrical muscle stimulation (EMS) suit apparatus, the apparatus comprising:
a garment comprising an elastic material that is configured to conform to a user's body;
a plurality of electrode assemblies on an inner face of the garment, the electrode assemblies each configured to sit flush against the user's skin;
a plurality of electrical connectors electrically coupling the electrode assemblies to controller for applying EMS; and
a controller comprising a compliance monitoring module, where the compliance monitoring module is configured to track the application of energy to the user's skin over a compliance period and to output compliance data based.

18.-59. (canceled)
